(19) United States
(12) Patent Application Publication
(10) Pub. No.: US 2014/0286998 A1
(43) Pub. Date: Sep. 25, 2014

(54) Double-Stranded RNA For Immunostimulation

(71) Applicant: RiboxX GmbH, Radebeul (DE)

(72) Inventor: Jacques Rohayem, Dresden (DE)

(73) Assignee: RIBOXX GMBH, Radebeul (DE)

(21) Appl. No.: 14/355,528

(22) PCT Filed: Oct. 31, 2012
(86) PCT No.: PCT/EP2012/071640

§ 371 Date: Apr. 30, 2014

Related U.S. Application Data
(60) Provisional application No. 61/581,983, filed on Dec. 30, 2011.

(30) Foreign Application Priority Data
Oct. 31, 2011 (EP) 11187345.1
Dec. 30, 2011 (EP) 11196165.2

(51) Int. Cl. C12N 15/117

(52) U.S. Cl. CPC C12N 15/117
USPC 424/278.1; 536/23.1

(57) ABSTRACT

The present invention relates to a ribonucleic acid (RNA) of double-stranded structure which is capable of triggering Toll-like receptor 3 (TLR-3) and which shows an increased serum stability while simultaneously being unable to be processed by the DICER complex.

### FIELD OF THE INVENTION

The present invention relates to a ribonucleic acid (RNA) of double-stranded structure which is capable of triggering Toll-like receptor 3 (TLR-3) and which shows an increased serum stability while simultaneously being unable to be processed by the DICER complex.

### BACKGROUND OF THE INVENTION

It is known that double-stranded RNA causes immunostimulation through the TLR-3 located in endosomes of dendritic and epithelial cells (see Kucnick et al. (2010) Current Medical Chemistry; Coffmann et al. (2010) Immunity).

WO 2008/014979 A2 and WO 2009/095226 A2 disclose nucleic acids of the general formula GₗXₘGₙ or CₗXₘCₙ and (NᵤGₗXₘGₙNᵥ)ₐ or (NᵤCₗXₘCₙNᵥ)ₐ, respectively. According to this prior art, such nucleic acids can be double-stranded RNA and have immunostimulative properties. However, specific examples of molecules according to the above formulae disclosed in WO 2008/014979 A2 and WO 2009/095226 A2 are single-stranded and have highly artificial sequences containing stretches of G or C nucleotides at both ends and a polyU stretch or a repeat containing C or G and U nucleotides between the C or G, respectively, ends such as the following:

(SEQ ID NO: 1)
GGGGGGGGUUUUUUUUUUUUUUUUUUUUUUUUUUUUUUGGGG
GGG;
(SEQ ID NO: 2)
GGGGGGGGGUUUUUUUUUUUUUUUUUUUUUUUUUUUUUUGGG
GGGGG;
(SEQ ID NO: 3)
GGGUUUUUUUUUUUUUUUGGGUUUUUUUUUUUUUUUGGGUUUU
UUUUUUUUUUUGGG;
(SEQ ID NO: 4)
GGGUUUGGGUUUGGGUUUGGGUUUGGGUUUGGGUUUGGGUUU
GGGUUUGGG;
(SEQ ID NO: 5)
CCCUUUUUUUUUUUUUUUCCCUUUUUUUUUUUUUUUCCCUUUU
UUUUUUUUUUUCCC;
(SEQ ID NO: 6)
CCCUUUCCCUUUCCCUUUCCCUUUCCCUUUCCCUUUCCCUUUC
CCUUUCCC;
and
(SEQ ID NO: 7)
CCCCCCCCCCCCCCCCCCCCGGUUUUUUUUUUUUUUUGGG.

The technical problem underlying the present invention is to provide an improved system for immunostimulation using RNA molecules.

### SUMMARY OF THE INVENTION

The solution to the above technical problem is provided by the embodiments described herein and in the claims.

In particular, the present invention provides a ribonucleic acid which has at least a segment of double-strand structure of a minimum of 45 bp wherein said double-stranded segment has increased thermodynamic stability on both ends (or end regions) of the dsRNA or dsRNA segment, respectively.

More particularly, it is provided a ribonucleic acid comprising at least one segment of double-stranded structure of at least 45 bp, preferably of at least 48 bp, more preferably of at least 70 bp, particularly preferred of from 45, 46, 47 or 48 to 200 bp or even more bp, wherein said at least one segment of double-stranded structure has a first and a second end each having at least 3 to 10 (preferably consecutive) G/C by within the last 6 to 20, respectively, by calculated from the last by of the respective end of the double-stranded structure. Due to the increased presence of G/C base pairing at both ends or in both end regions of the double-stranded structure it results a thermodynamic “clamp” providing the RNA of the invention with a surprisingly high serum-stability and stability against cellular RNA degrading enzymes such as DICER. Accordingly, the inventive RNAs cannot be processed by cellular RNAses like the DICER complex. Therefore, the ribonucleic acid of the present invention displays a superior triggering of the TLR-3 pathway leading to a sustained and powerful immunostimulation (innate immune response).

According to the present invention “3 to 10 G/C by within the last 6 to 20 bp” at each end of the double-stranded ribonucleic acid or of the double-stranded segment of the ribonucleic acid, respectively, typically means that at least 50% of the last 6 to 20 bp at each end of the double-stranded structure are G/C base pairs, preferably the G/C base pairs are consecutive. Accordingly, at least 3 G/C base pairs are present within the last 6 bp, at least 4 G/C base pairs are present within the last 8 bp, at least 5 G/C base pairs are present within the last 10 bp and so on.

The ribonucleic acid according to the invention has a nucleotide sequence between the last 6 to 20 bp at each end that is heteropolymeric, wherein ribonucleic acids having the following sequences are excluded:

(SEQ ID NO: 3)
GGGUUUUUUUUUUUUUUUGGGUUUUUUUUUUUUUUUGGGUUUU
UUUUUUUUUUUGGG
(SEQ ID NO: 4)
GGGUUUGGGUUUGGGUUUGGGUUUGGGUUUGGGUUUGGGUUU
GGGUUUGGG
(SEQ ID NO: 5)
CCCUUUUUUUUUUUUUUUCCCUUUUUUUUUUUUUUUCCCUUUU
UUUUUUUUUUUCCC
(SEQ ID NO: 6)
CCCUUUCCCUUUCCCUUUCCCUUUCCCUUUCCCUUUCCCUUU
CCCUUUCCC

Preferably, the at least 3 to 10 terminal by of each end of the double-stranded structure in the ribonucleic acid of the invention are G/C bp.

Thus, in other words, the ribonucleic acid or double-stranded segment of the nucleic acid of the present invention has a nucleotide strand that can be represented by the following general formula (1):

(X)₆₋₂₀(N)ₐ(X)₆₋₂₀

wherein X is any nucleotide with the proviso that at least 3 to 10 nucleotides are G and/or C nucleotides, N is any nucleotide and a is selected such that the ribonucleic acid (or double-stranded segment thereof) has a length of at least 45 bp wherein the sequence of N is heteropolymeric, and wherein the other strand of the ribonucleic acid or the double-stranded segment of the ribonucleic acid has a complementary sequence to the above defined sequence.

It is not necessary that the ribonucleic acid of the present invention is completely double-stranded. However, typically it is essentially double-stranded, i.e. there is regular hydrogen bonding between the opposite bases of the strands forming the double-stranded structure, but there may be one or several mismatches through non-complementary bases or through chemical modifications. Usually, such mismatches do not exceed 10%, more preferably 5%, particularly preferred 2% or even 1% of the double-stranded molecule (or double-stranded segment of the inventive RNA, respectively).

According to preferred embodiments, the ribonucleic acid of the invention containing the double-stranded structure may also have overhangs at one or both ends of the double-stranded structure which can be of various length but typically do not exceed ten single-stranded residues, more preferably such overhangs comprise one, two or three residues. These overhangs may also comprise deoxyribonucleotides instead of ribonucleotides.

The ribonucleic acid of the present invention containing a double-stranded structure may be composed of one polyribonucleic strand that has a sequence allowing the formation of a hairpin structure such that the double-stranded structure as defined before builds up under appropriate conditions. However, in other preferred embodiments the ribonucleic acid according to the present invention comprises two separate polyribonucleotide strands.

According to preferred embodiments of the ribonucleic acid of the invention the strands of the at least one segment of double-stranded structure have the following sequences:

5′-(G/C)ₓ(N)_{y}(G/C)_{z}-3′

3′-(G/C)ₓ(N)_{y}(G/C)_{z}-5′

In the above structure G/C is a G or C ribonucleotide with the proviso that when a G ribonucleotide is in a position in one strand the other strand has a C ribonucleotide in the opposite position and vice versa; N is any ribonucleotide with the proviso that the sequences of both strands are essentially complementary; each x is an integer of from 3 to 10, in particular 3, 4, 5, 6, 7, 8, 9, or 10, each y is typically an integer of 30 to 200 or more such as 500 or 600, preferably, 30 to 150, more preferably 30 to 100, even more preferred 30 to 50, and each z is an integer of from 3 to 10, with the proviso that x in one strand is equal to the x in the other strand, y in one strand is equal to the y in the other strand, and z in one strand is equal to the z in the other strand; and with the further proviso that the length of said double-stranded structure is at least 45 bp, typically 45 to 520 bp or more such as 620 bp, preferably 45 to 220 bp, more preferably 45 to 150 bp, particularly preferred 45 to 90 bp, even more preferred 45 to 70 bp.

More preferably, the strands of the at least one segment of double-stranded structure are one of the following sequence pairs:

5′-(G)₅(N)_{y}(G)₅-3′

3′-(C)₅(N)_{y}(C)₅-5′

or

5′-(C)₅(N)_{y}(C)₅-3′

3′-(G)₅(N)_{y}(G)₅-5′

or

5′-(G)₅(N)_{y}(C)₅-3′

3′-(C)₅(N)_{y}(G)₅-5′

or

5′-(C)₅(N)_{y}(G)₅-3′

3′-(G)₅(N)_{y}(C)₅-5′

wherein y is typically an integer of from 35 to 200 or more such as 500 or 600, preferably 35 to 150, more preferably 35 to 100, particularly preferred 35 to 80, even more preferred 35 to 40.

The sequence of the double-stranded region between the G/C “clamps” of the ribonucleic acid of the invention is in principle not critical, however, homopolymers of any of the ribonucleobases A, U, G, or C, in particular G or C, are not well accepted due to their potential toxicity. Normally, the sequence is chosen such that the overall sequence shows no evident complementarities to other existing RNA or DNA species in a cell or organism, particularly in order to avoid potential side effects from posttranscriptional gene silencing mechanisms. However, it may be appropriate that the sequence between the C/C base pairs at both ends, or generally speaking, between the G/C clamps as defined above, may be assembled from naturally occurring (partial) sequences that may be repeated. An example is a certain (partial) sequence of, e.g. 20 to 90 nucleotides, of a naturally occurring sequence, e.g. of a virus, microorganism or other organism, that is repeated once or several times such as two, three, four or five or even more times, in order to provide a molecule of the present invention having the appropriate or desired, respectively, length.

In contrast thereto, with regard to an easier and less expensive production and also in order to avoid highly artificial and potentially toxic sequences such as those disclosed in WO 2008/014979 and WO 2009/095226, it is preferred that the double-stranded region between the G/C clamps, is free of homopolymeric stretches of more than 5 contiguous identical nucleotides, and is free of short repeated sequence motifs of 3 to 10 nucleotides per motif.

Furthermore, in order to further increase the stability of the RNA of the invention, the G/C content of the double-stranded structure (comprising the G/C clamps) is at least 45%, more preferably 45 to 70%, particularly preferred 48 to 60%. It is also contemplated that the sequence between the “clamps” contains further (short) stretches of G/C base pairs, e.g. 3, 4 or 5 G/C base pairs.

Such embodiments of the invention usually have a Tm of the double-stranded structure of from 68 to 80 C.

In consideration of production costs to effective triggering of TLR-3 (dimerization), preferred lengths of the dsRNA structure, more preferably of the complete RNA, range from 45 to 150 bp, particularly preferred from 45 to 100 bp, even more preferred from 45 to 50 bp, i.e. 45, 46, 47, 48, 49 or 50 bp.

Specific preferred examples of the double-stranded RNA according to the invention are composed of the following (complementary) sequence pairs:

Riboxxim ®-1:
(SEQ ID NO: 8)
5′-CCCCCUAAGCACGAAGCUCAGAGUUAAGCACGAAGCUCAGA
GUCCCCC-3′
(SEQ ID NO: 9)
5′-GGGGGACUCUGAGCUUCGUGCUUAACUCUGAGCUUCGUGCUUA
GGGGG-3′
Riboxxim ®-2:
(SEQ ID NO: 10)
5′-CCCCCGAACGAAUUUAUAAGUGGGAACGAAUUUAUAAGUGGCCC
CC-3′
(SEQ ID NO: 11)
5′-GGGGGCCACUUAUAAAUUCGUUCCCACUUAUAAAUUCGUUCGG
GGG-3′
Riboxxim ®-3:
(SEQ ID NO: 12)
5′-CCCCCACAACAUUCAUAUAGCUGACAACAUUCAUAUAGCUGCCC
CC-3′
(SEQ ID NO: 13)
5′-GGGGGCAGCUAUAUGAAUGUUGUCAGCUAUAUGAAUGUUGUGG
GGG-3′
dsRNA-90:
(SEQ ID NO: 14)
5′-CCCCCUAAGCAGCAAGCCUCAGCAGCUAAGCCAGCAGCCUCAGC
AGCUAGCAGCAAGCUCAGCAGCUAAGCCACGAGCUCAUGCGCC
CCC-3′
(SEQ ID NO: 15)
5′-GGGGGCGCAUGAGCUCGUGGCUUAGCUGCUGAGCUUGCUGCU
AGCUGCUGAGGCUGCUGGCUUAGCUGCUGAGGCUUGCUGCUUA
GGGGG-3′

As mentioned before, the RNA of the present invention may have overhangs on one or both sides, but it is typically blunt-ended on both sides of the double-strand structure.

It is contemplated that the RNA of the present invention is equipped with further entities to improve the immunostimulative properties. For example, it would be possible to include CpG deoxyribonucleotide motifs into the ribonucleic acid of the invention (one or more of them) in order to trigger the Toll-like receptor 9 (TRL-9), at least partially.

Further particularly preferred ribonucleic acids of the invention have a free triphosphate group at the 5′-end of at least one strand. Such triphosphate-containing RNAs of the invention are preferably completely double-stranded and one or both 5′ ends contain(s) a free triphosphate group. A “free triphosphate group” in this context means that this triphosphate group is not modified and specifically does not contain, or is not part of, respectively, a cap structure. Such triphosphate-containing RNAs of the invention are particularly potent immunostimulative compounds. Especially preferred examples of ribonucleic acids according to the invention containing a triphosphate group are the following sequence pairs (the complementary strands are given in 5′ to 3′ orientation):

Riboxxim ®-48:
(SEQ ID NO: 8)
5′-CCCCCUAAGCACGAAGCUCAGAGUUAAGCACGAAGCUCAGAGUC
CCCC-3′
(SEQ ID NO: 9)
PPP-5′-GGGGGACUCUGAGCUUCGUGCUUAACUCUGAGCUUCGUGCUUA
GGGGG-3′
Riboxxim ®-90:
(SEQ ID NO: 14)
5′-CCCCCUAAGCAGCAAGCCUCAGCAGCUAAGCCAGCAGCCUCAGC
AGCUAGCAGCAAGCUCAGCAGCUAAGCCACGAGCUCAUGCGCC
CCC-3′
(SEQ ID NO: 15)
ppp-5′-GGGGGCGCAUGAGCUCGUGGCUUAGCUGCUGAGCUUGCUGCU
AGCUGCUGAGGCUGCUGGCUUAGCUGCUGAGGCUUGCUGCUUA
GGGGG-3′

wherein ppp denotes the free 5′-triphosphate group.

A free triphosphate-containing double-stranded ribonucleic acid of the present invention triggers both RIG-I (see. e.g. WO 2008/017473 A2) and TLR-3 pathways and exerts a dose-dependent innate immune response as demonstrated in the examples.

The RNA of the present invention may also contain one or more modified nucleotide analogues, in particular based upon stability considerations.

The chemical modification of the nucleotide analogue in comparison to the natural occurring nucleotide may be at the ribose, phosphate and/or base moiety. With respect to molecules having an increased stability, especially with respect to RNA degrading enzymes, modifications at the backbone, i.e. the ribose and/or phosphate moieties, are especially preferred.

Preferred examples of ribose-modified ribonucleotides are analogues wherein the 2′-OH group is replaced by a group selected from H, OR, R, halo, SH, SR, NH₂, NHR, NR₂, or CN with R being C₁-C₆ alkyl, alkenyl or alkynyl and halo being F, Cl, Br or I. It is clear for the person skilled in the art that the term “modified ribonucleotide” also includes 2′-deoxyderivatives, such as 2′-O-methyl derivatives, which may at several instances also be termed “deoxynucleotides”.

As mentioned before, the at least one modified ribonucleotide may be selected from analogues having a chemical modification at the base moiety. Examples of such analogues include, but are not limited to, 5-aminoallyl-uridine, 6-aza-uridine, 8-aza-adenosine, 5-bromo-uridine, 7-deaza-adenine, 7-deaza-guanine, N⁶-methyl-adenine, 5-methyl-cytidine, pseudo-uridine, and 4-thio-uridine.

Examples of backbone-modified ribonucleotides wherein the phosphoester group between adjacent ribonucleotides is modified are phosphothioate groups.

The present invention also provides a method of producing the RNA as defined herein comprising the steps of:
(i) synthesizing a ribonucleic acid having a sequence that folds into a form such that it adopts the double-stranded structure as defined above; or
(ii) synthesizing a first ribonucleic acid strand and a second ribonucleic acid strand, and annealing the two strands under hybridization conditions wherein the strands have the appropriate sequences so as to form the double-stranded structure as defined herein.

The polynucleotide molecules for providing the double-stranded RNA of the present invention may be prepared by chemical synthesis methods or enzymatically, or by a combination of chemical and enzymatic steps. Methods for the enzymatic preparation of the double-stranded RNA molecules of the present invention are preferably those that use RNA-dependent RNA polymerases of caliciviruses as disclosed in WO-A-2007/012329. With respect to enzymatic synthesis of chemically modified dsRNAs using RNA-dependent RNA polymerases, the methods referred to in WO 2009/150156 are preferred. Chemical synthesis methods for preparing RNA strands are also well-known in the art.

The present invention also contemplates a vector encoding RNA species as defined herein. For example, a single-stranded dsRNA-containing entity as disclosed herein may be expressed in the form of a shRNA derivative.

Appropriately, double-stranded RNAs of the invention can also be labelled (for example, if there is an overhang on one side of the double-strand structure) for detection in research or diagnostic applications. The “label” can be any chemical entity which enables the detection of the RNA in question via physical, chemical and all biological means. Examples of typical labels linked to or integrated into one or more of the nucleotides or added to one end of the molecules as disclosed herein are radioactive labels, chromophores and fluorophores (e. g. fluorescein, TAM etc.).

The ribonucleic acids of the present invention are particularly for use as agonists of TLR-3. Further preferred ribonucleic acids of the invention containing a free 5′-triphosphate moiety are also agonists of RIG-I. In that function, the RNA molecules of the present invention exert an immunostimulatory effect in cells or organisms. Ribonucleic acids of the present invention are therefore useful as medicaments, in particular immunostimulatory preparations. Ribonucleic acids of the present invention are also contemplated for the manufacture of a medicament for immunostimulation. The present invention is therefore also directed to a pharmaceutical composition comprising at least one ribonucleic acid as defined herein in combination with at least one pharmaceutically acceptable carrier, excipient, and/or diluent. The preparation of pharmaceutical compositions in the context of the present invention, their dosages and their routes of administration are known to the skilled person, and guidance can be found in the latest edition of Remington's Pharmaceutical Sciences (Mack publishing Co., Eastern, PA, USA). It is also contemplated that the pharmaceutical compositions of the invention contain two or more different RNAs as defined herein.

In cases where the RNA of the invention is used as an immunostimulatory drug alone, a topical administration of the appropriate preparation (e.g. a spray) to skin and/or mucosa is preferred. As an “RNA drug” the RNA of the present invention leads to stimulation of dendritic cells and generation of a CD8+ T cell response. Such application of the inventive dsRNA is especially useful in the treatment of infectious diseases, e.g. by viruses (such as Herpesvirus, Papillomavirus) or in the treatment of cancer.

The improved immunostimulatory effects the ribonucleic acids of the present invention are also useful in combination with a vaccine directed to a certain disease. Thus, the RNA of the invention may also be used as an “RNA adjuvant” in vaccine preparations (or administered in a distinct preparation together with the vaccine, or sequentially). Simultaneous or sequential administration of the vaccine and the immunostimulatory RNA of the present invention should improve the immune response against the antigen of the vaccine by generating a protective CD8+ T cell response to soluble proteins (antigens), triggering DC activation and induction of type I IFN production.

The ribonucleic acids or pharmaceutical compositions as disclosed herein can also be combined with further immunostimulatory drugs known in the art.

The ribonucleic acids of the present invention are particularly useful in the treatment of diseases, including infectious diseases caused by infectious agents such as a bacterium, virus, and fungus, and tumours.

The present invention also provides a method for the treatment of a disease as mentioned above, preferably a viral infection or a tumour disease, comprising administering an effective amount of the pharmaceutical composition of the invention to a preferably mammalian, particularly human, patient in need of such treatment.

The present invention also relates to a cell or non-human organism being transfected or transformed with the double-stranded RNA molecule as defined herein or with a vector coding therefor.

### BRIEF DESCRIPTION OF THE DRAWINGS

The figures show:

FIGS. 1A-1B show a native polyacrylamide gel (20%) after ethidium bromide staining of reactions demonstrating that an exemplary ribonucleic acid of the invention (Riboxxim®-1) is stable for at least 72 h in 80% FCS at 37° C. A reaction containing no FCS was used as a negative control.

FIGS. 2A-2B show that ribonucleic acids of the present invention are resistant to human DICER. (A) Sequences of the strands in the double-stranded molecules in the exemplary RNAs of the invention called Riboxxim®-1, Riboxxim®-2 and Riboxxim®-3: length (in base pairs), Tm and G/C content are indicated. (B) Native polyacrylamide gel (20%) after ethidium bromide staining of reactions containing 1.6 μM of Riboxxim®-1, -2 or -3 and recombinant human DICER incubated at 37° C. for 12 h. It can be seen that Riboxxim®-1 Riboxxim®-2 and Riboxxim®-3 are not degraded by human DICER. dsRNA marker of 17 bp, 21 bp and 25 bp, and a ssRNA marker of 24 nt are indicated.

FIG. 3 shows the sequences of the strands of double-stranded RNA molecules with a triphosphate moiety at one 5′ end according to the invention called Riboxxim®-48 and Riboxxim®-90 as well as the sequence of a double-stranded RNA molecule without a triphosphate moiety at one 5′ end (dsRNA-90); length (in base pairs), Tm, G/C content and the presence of the triphosphate moiety are indicated.

FIG. 4 shows a photograph of an ethidium bromide-stained 10% polyacrylamide gel after electrophoretic separation of the indicated nucleic acids demonstrating their length. Left panel: An RNA marker corresponding to dsRNA of 100 bp is shown in the first lane from the left, and an RNA marker corresponding to dsRNA of 25 bp, 21 bp and 17 bp is shown in the fourth lane from the left. RNAs according to the invention Riboxxim®-90 and Riboxxim® 48 are shown in the second and third lane, respectively, from the left. Right panel: the first lane from the left shows an RNA marker corresponding inter alia to dsRNA of 100 bp, as indicated. The second and third lane from the left show dsRNA-90 and Riboxxim®-90, respectively.

FIGS. 5A-5B show two diagrams indicating the concentration of the cytokine IL-6 in the cell culture supernatant of dendritic cells (slanDC, CD1c+) and monocytes due to secretion of the cytokine by the indicated cells after stimulation with double-stranded RNA according to the invention bearing a triphosphate moiety, Riboxxim®-48 (FIG. 5A) or Riboxxim®-90 (FIG. 5B).

FIG. 6 shows a diagram indicating the amount of secreted IL-6 after stimulation of myeloid dendritic cells (CD1c+) and monocytes with a double-stranded RNA according to the invention (dsRNA-90).

FIGS. 7A-7C show diagrams of the concentration of IL-1β in the cell culture supernatant of slan dendritic cells (FIG. 7A), myeloid dendritic cells (CD1c+, FIG. 7B) and monocytes (FIG. 7C) indicating a dose-dependent secretion of IL-1β after stimulation with Riboxxim®-90 in contrast to a dose-independent, generally high secretion of IL-1β after stimulation with poly(I:C). The cells were stimulated with 6.25 μg/ml, 12.5 μg/ml, 25 μg/ml, and 50 μg/ml of the respective nucleic acid.

FIGS. 8A-8C show diagrams of the concentration of IL-6 in the cell culture supernatant of slan dendritic cells (FIG. 8A), myeloid dendritic cells (CD1c+, FIG. 8B) and monocytes (FIG. 8C) indicating a dose-dependent secretion of IL-6 after stimulation with Riboxxim®-90 in comparison to the secretion of IL-6 after stimulation with poly(I:C). The cells were stimulated with 6.25 μg/ml, 12.5 μg/ml, 25 μg/ml, and 50 μg/ml of the respective nucleic acid.

FIGS. 9A-9B show diagrams of the concentration of IL1β (FIG. 9A) and IL-6 (FIG. 9B), respectively, in the cell culture supernatant of monocytes isolated from two different donors indicating a dose-dependent secretion of the respective interleukin after stimulation with triphosphated Riboxxim®-90 or not triphosphated dsRNA90 in comparison to the stimulation with poly(I:C). The mean values+/−SEM of at least three independent measurements per blood donor are shown.

FIGS. 10A-10B show the results of an electrospray ionization mass spectrometry of a double-stranded RNA-molecule bearing a triphosphate moiety at one 5′ end, Riboxxim®-48; The MS-analysis of the antisense strand, shown in FIG. 10A, corresponds to the calculated mass of the strand with the following sequence:
5′-CCCCCUAAGCACGAAGCUCAGAGUUAAGCACGAAGCUCAGAGUC CCCC-3′ (SEQ ID NO: 8). The different ionization states are depicted. The MS-analysis of the sense strand, shown in FIG. 10B, corresponds to the calculated mass of the strand with the following sequence:

(SEQ ID NO: 9)
PPP-5′-GGGGGACUCUGAGCUUCGUGCUUAACUCUGAGCUUCGUGCUUA
GGGGG-3′.

This strand bears a triphosphate moiety at its 5′-end. The different ionization states are shown.

### DETAILED DESCRIPTION OF THE INVENTION

The present invention is further illustrated by the following non-limiting examples.

### EXAMPLES

### Example 1

### Serum Stability of dsRNAs of the Invention

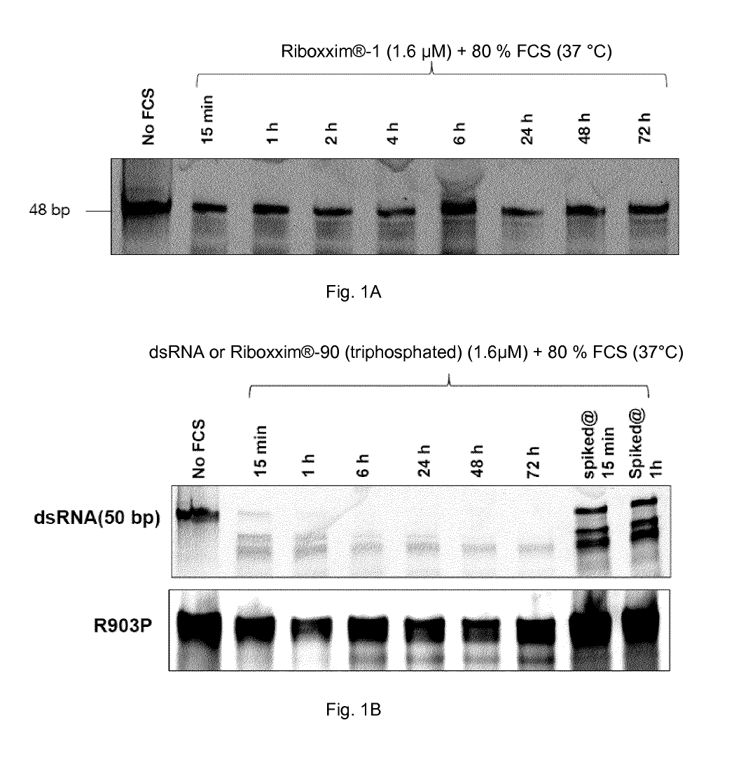

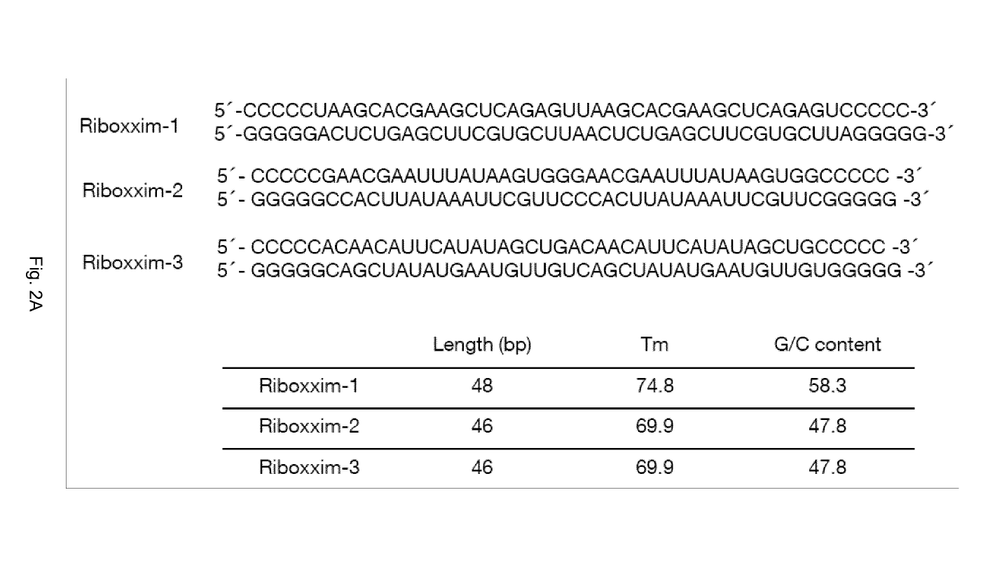

A reaction with a total volume of 50 μl contained 0.08 nmole double-stranded RNA species (Riboxxim®-1, see FIG. 1A; Riboxxim®-90, see FIG. 1B; control dsRNA of 50 bp having an arbitrary sequence, see FIG. 1B) and 80% fetal calf serum (FCS). The reaction was incubated at 37° C. At the time points indicated in FIGS. 1A and 1B, respectively (15 min; 1 h; 2 h; 4 h; 6 h; 24 h; 48 h; 72 h) 5 μl of the reaction was withdrawn at each time point and frozen at −80° C. As a further control, the RNA was spiked in the reaction at the indicated time points (15 min and 1 h) in FIG. 1B. The reaction products at the above time points were separated on a native 20% polyacrylamide gel and the bands visualized by UV transillumination after ethidium bromide staining.

### Example 2

### Resistance of dsRNAs of the Invention to Human DICER

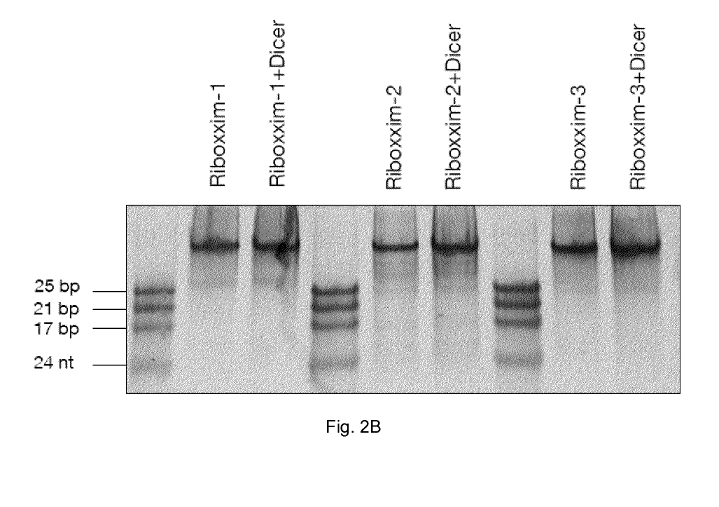

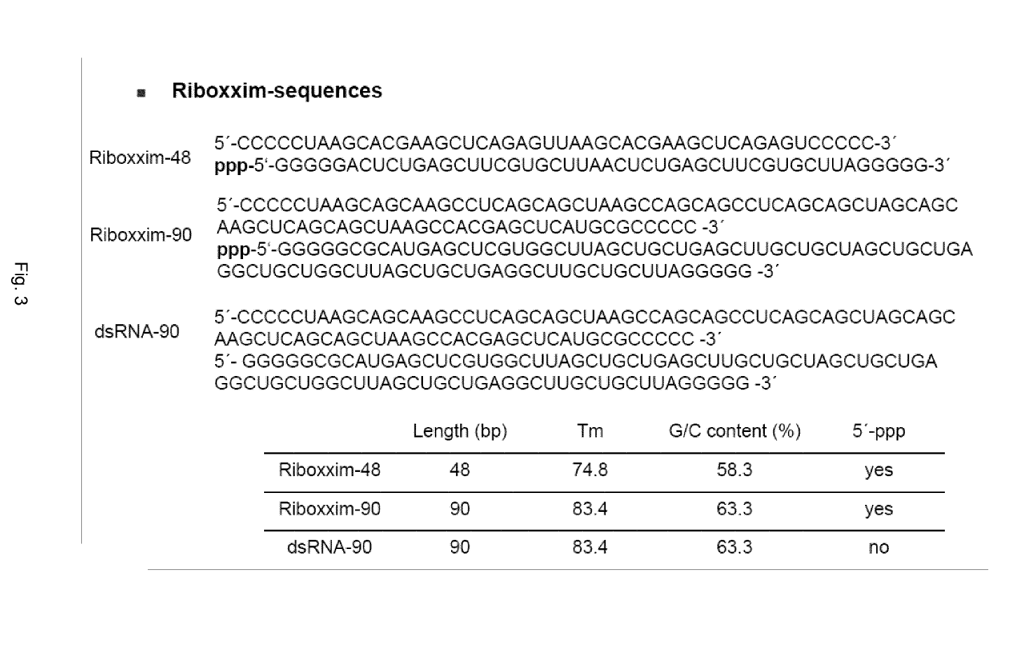

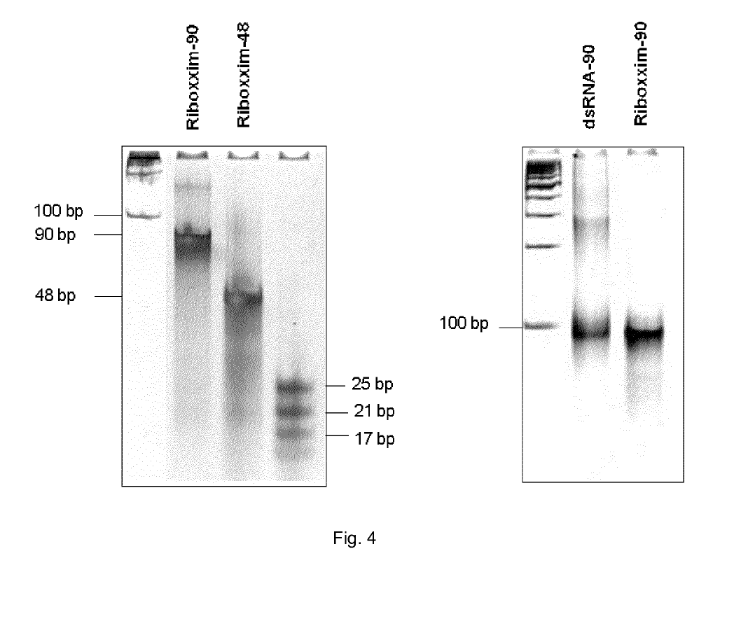

The dsRNA molecules indicated in FIG. 2A were incubated in a total volume of 15 μl containing 250 ng of the respective double-strained RNA (Riboxxim®-1, Riboxxim®-2 or Riboxxim®-3), 2 μl recombinant human DICER enzyme (1U), 4 μl DICER reaction Buffer, 0.5 μl of 50 mM MgCl₂, 1 μl of 10 mM ATP, and RNAse-DNAse-free water to a final volume of 15 μl. The reaction was incubated at 37° C. for 12 hours, then separated on a native 20% polyacrylamide gel and visualized through UV transillumination after ethidium bromide staining.

FIG. 2B shows the results of incubation with the human DICER complex. Neither of the ribonucleic acids of the invention can be processed by DICER.

### Example 3

### Secretion of IL-6 after Stimulation with Double-Stranded RNA Molecule According to the Invention Having a Triphosphate Moiety at One 5′ End

Cells were isolated from the blood of two healthy donors. All cell populations were sorted on two columns via the autoMACS system according to the recommendation of the manufacturer (Miltenyi Biotech GmbH, Bergisch Gladbach, Germany). A detailed protocol for the isolation of 6-Sulfo LacNAc dendritic cells (slanDC) is described in Schaekel, K. et al. (1998); Eur. J. Immunol. 28, (p. 4084-4093). Briefly, myeloid dendritic cells (CD1c+) were isolated after purifying the slanDC; monocytes were isolated by a negative selection strategy. Isolated cells were grown in RPMI 1640 medium containing 10% human AB serum (CCPRO, Neustadt, Germany), 2 mM L-glutamine, 1% nonessential amino acids, 100 U/ml penicillin, and 100 mg/ml streptomycin (Biochrom AG, Berlin, Germany). Cells were then seeded in 96-well plates at a density of 25,000 cells/well in RPMI-1640 with 10% human AB serum. Subpopulations of dendritic cells or monocytes were stimulated with 50 μg/ml Riboxxim®-48 or Riboxxim®-90 for 24 to 72 hours. The concentration of secreted IL-6 in the culture supernatant was assessed using ELISA.

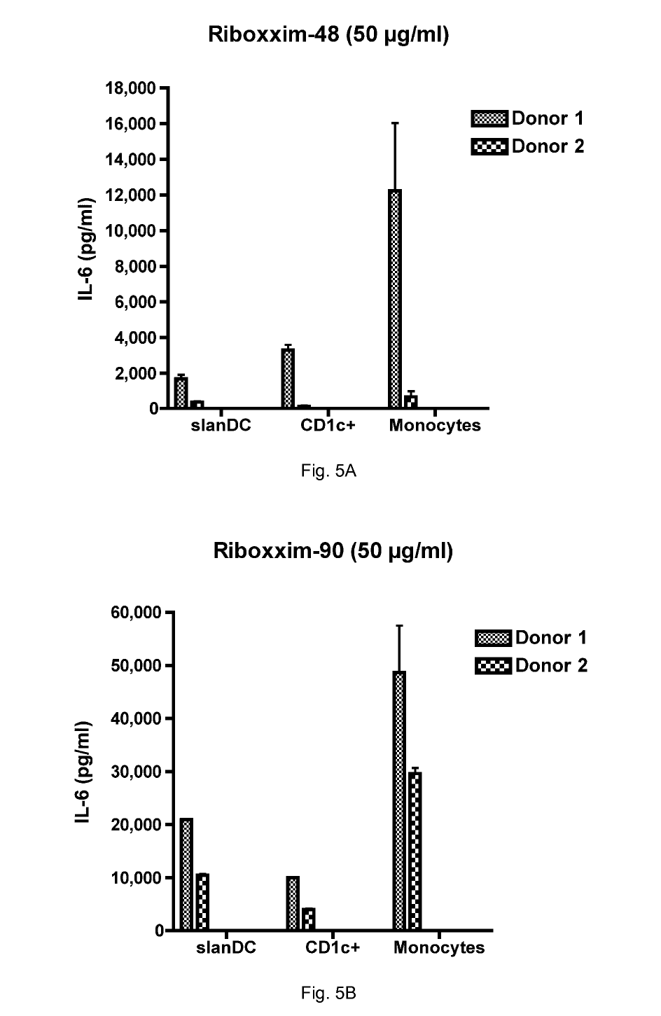

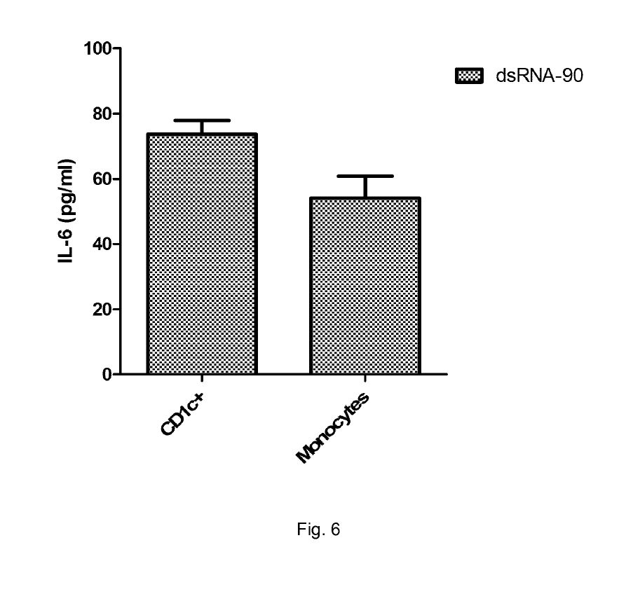

FIG. 5 shows the concentration of secreted IL-6 in the cell culture supernatant of dendritic cells and monocytes after incubation with Riboxxim®-48 (FIG. 5A) and Riboxxim®-90 (FIG. 5B), respectively. Both nucleic acids strongly stimulate the secretion of IL-6 in all cell types.

The data shown correspond to the mean values of three independent experiments with the respective standard error of the mean.

### Example 4

### Secretion of IL-6 in Response to a Double-Stranded RNA Molecule According to the Invention

Myeloid dendritic cells (CD1c+) and monocytes were isolated, sorted and grown as described in example 3. Cells were then seeded in 96-well plates at a density of 25,000 cells/well in RPMI-1640 with 10% human AB serum and incubated with 50 μg/ml double-stranded RNA (dsRNA-90) for 24 to 72 hours. The concentration of secreted IL-6 in the culture supernatant was analysed using ELISA.

FIG. 5 shows the concentration of IL-6 secreted by dendritic cells and monocytes after stimulation with double-stranded RNA without a triphosphate moiety at the 5′ end. The data shown correspond to the mean values of three independent experiments with the respective standard error of the mean.

### Example 5

### Secretion of IL-1β after Stimulation with Double-Stranded RNA According to the Invention Bearing a Triphosphate Moiety at One 5′ End is Dose-Dependent

Dendritic cells (slan DC and CD1c+) and monocytes were isolated, sorted and grown as described in example 3. Cells were then seeded in 96-well plates at a density of 25,000 cells/well in RPMI-1640 with 10% human AB serum and incubated with 6.25 μg/ml, 12.5 μg/ml, 25 μg/ml, or 50 μg/ml Riboxxim®-90 or poly(I:C) for 24 to 72 hours. The concentration of secreted IL-1β in the culture supernatant was assessed using ELISA.

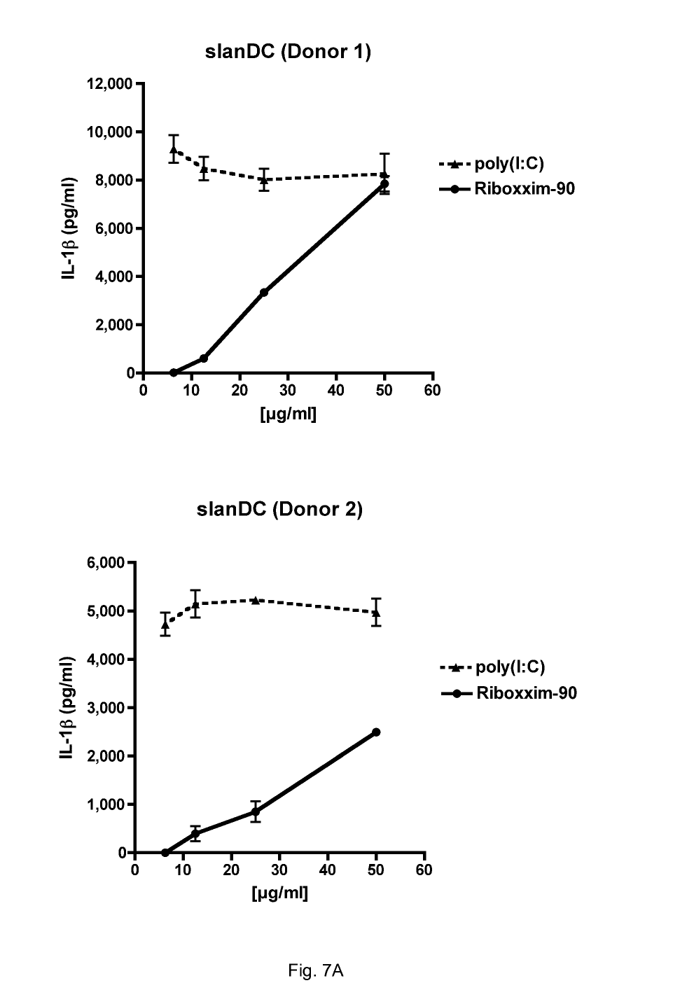

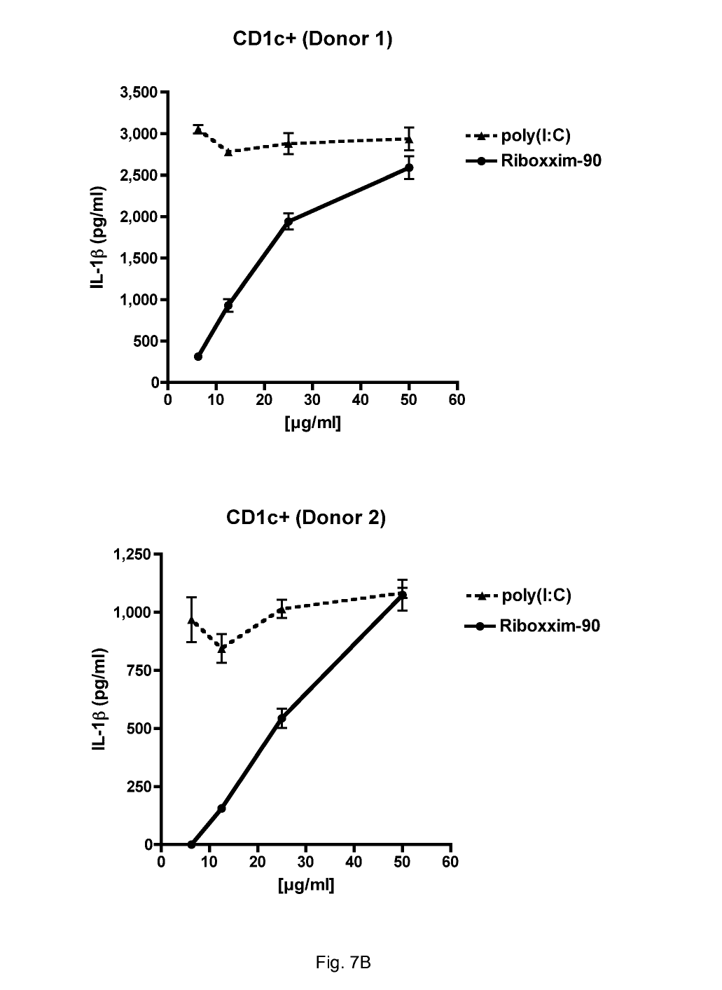

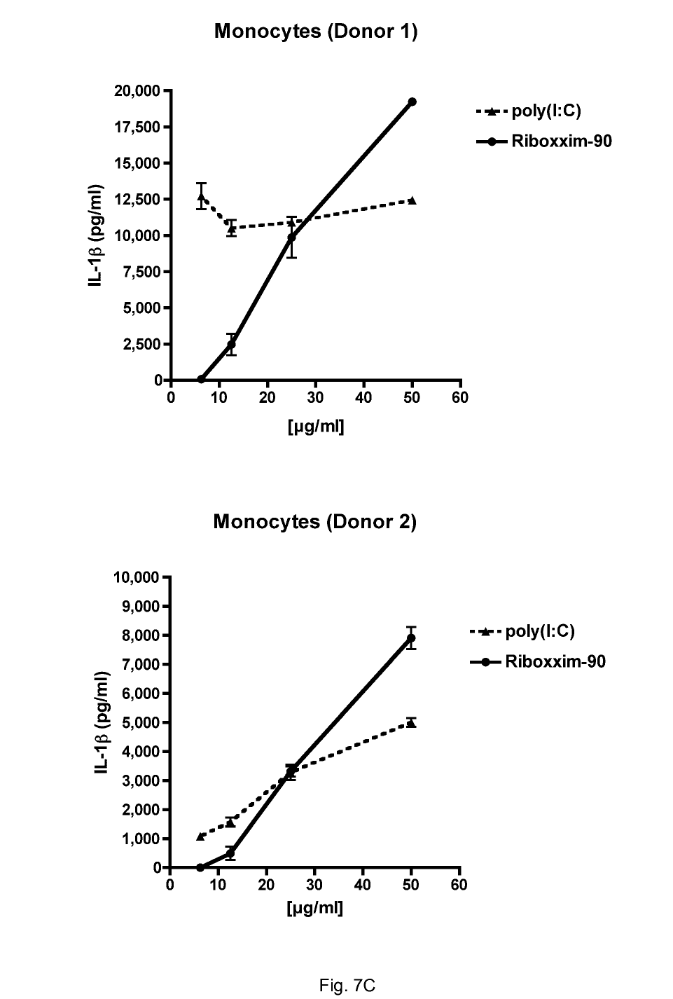

FIG. 7 shows the concentration of IL-1β secreted by slanDC (FIG. 7A), CD1c+ (FIG. 7B) and monocytes (FIG. 7C) after stimulation with increasing amounts of Riboxxim®-90 or poly(I:C). It is demonstrated that the secretion of IL-1β in response to Riboxxim®-90 is dose-dependent in all three cell types, whereas poly(I:C) triggers a dose-independent and generally high secretion of IL-1β, in particular in dendritic cells. The data shown correspond to the mean values of three independent experiments with the respective standard error of the mean.

### Example 6

### Secretion of IL-6 after Stimulation with Double-Stranded RNA According to the Invention Bearing a Triphosphate Moiety at One 5′ End is Dose-Dependent

Dendritic cells (slan DC and CD1c+) and monocytes were isolated, sorted and grown as described in example 3. Cells were then seeded in 96-well plates at a density of 25,000 cells/well in RPMI-1640 with 10% human AB serum and incubated with 6.24 μg/ml, 12.5 μg/ml, 25 μg/ml or 50 μg/ml Riboxxim®-90 or poly(I:C) for 24 to 72 hours. The concentration of secreted IL-6 in the culture supernatant was assessed using ELISA.

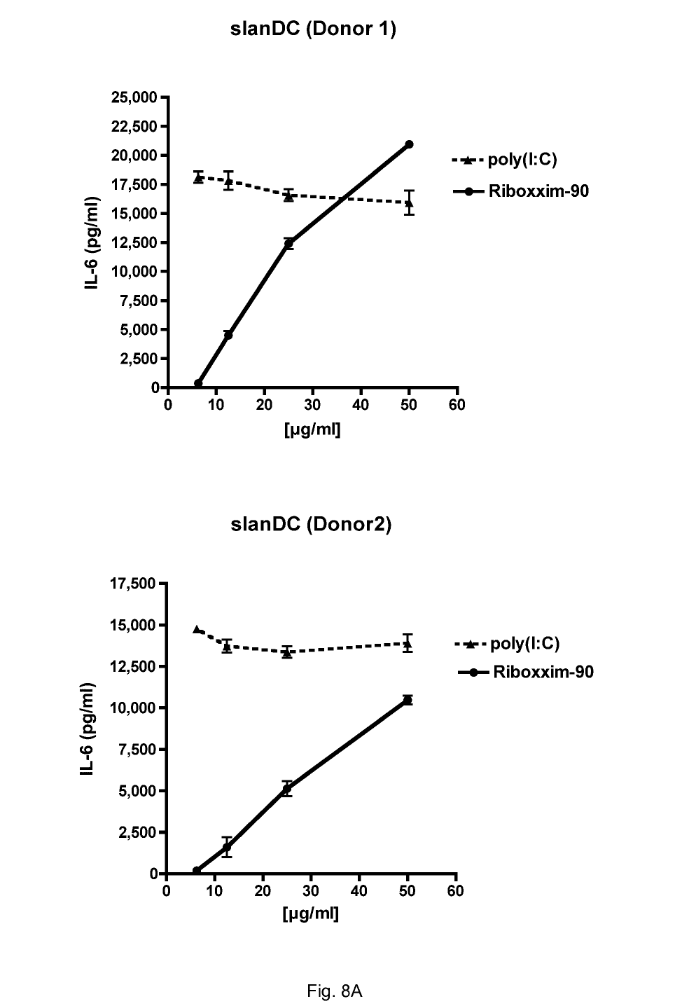

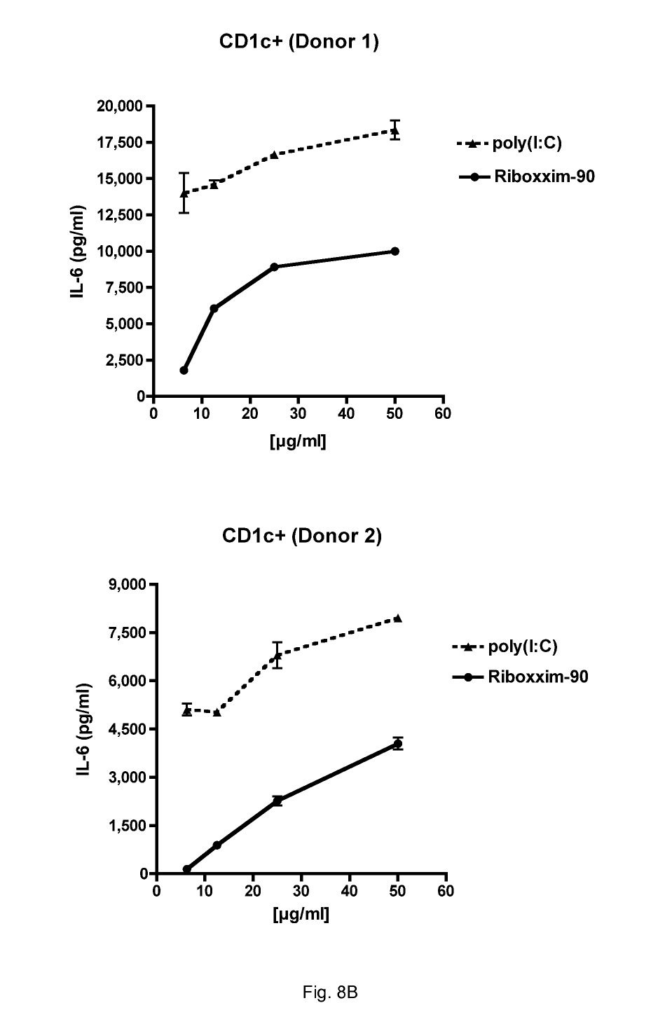

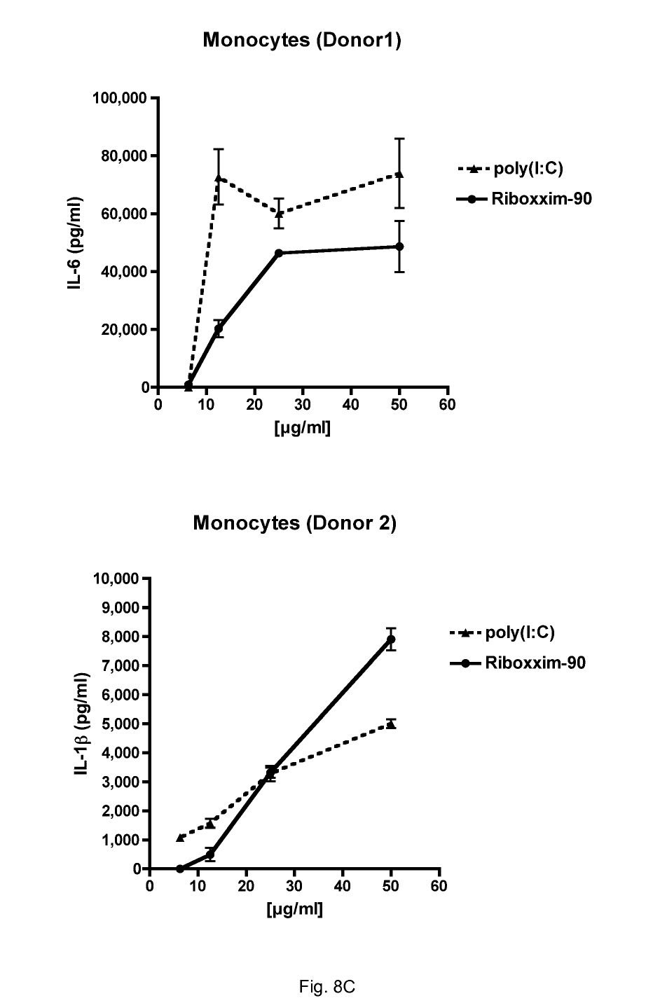

FIG. 8 shows the concentration of IL-6 secreted by slanDC (FIG. 8A), CD1c+ (FIG. 8B) and monocytes (FIG. 8C) after stimulation with increasing amounts of Riboxxim®-90 or poly(I:C). It is demonstrated that the secretion of IL-6 in response to Riboxxim®-90 is dose-dependent in all three cell types, whereas poly(I:C) triggers a dose-independent and generally high secretion of IL-6, in particular in dendritic cells. The data shown correspond to the mean values of three independent experiments with the respective standard error of the mean.

### Example 7

### Comparison of IL-1 and IL-6 Secretion after Stimulation with Double-Stranded RNA According to the Invention Bearing or not Bearing a Triphosphate Moiety at One 5′-End

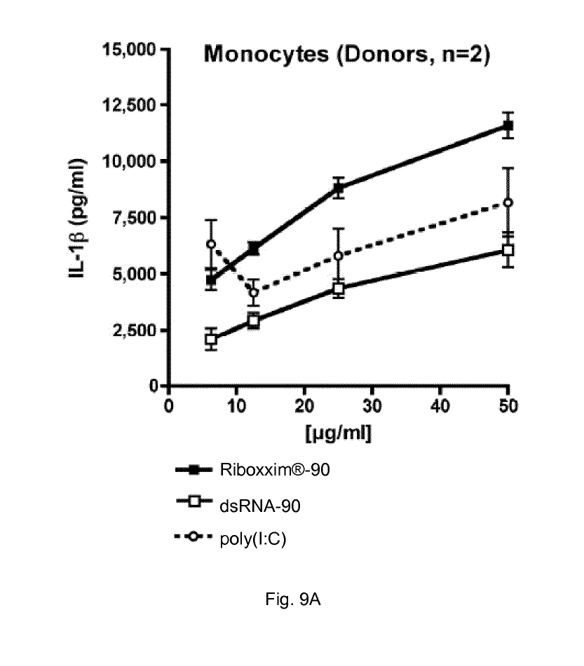

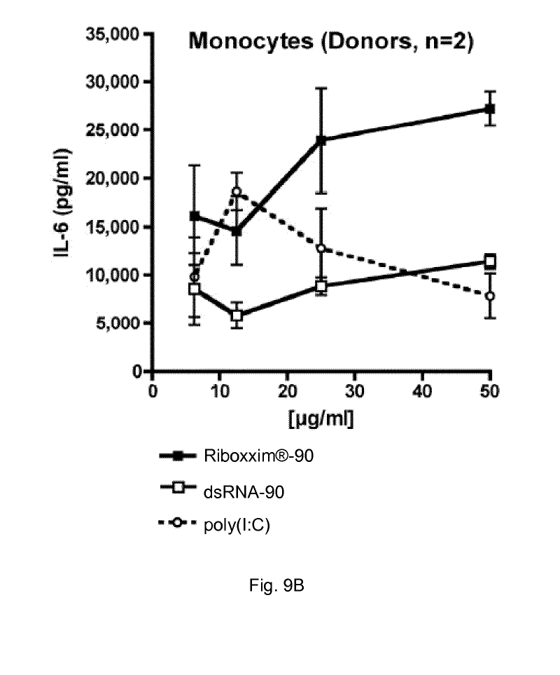

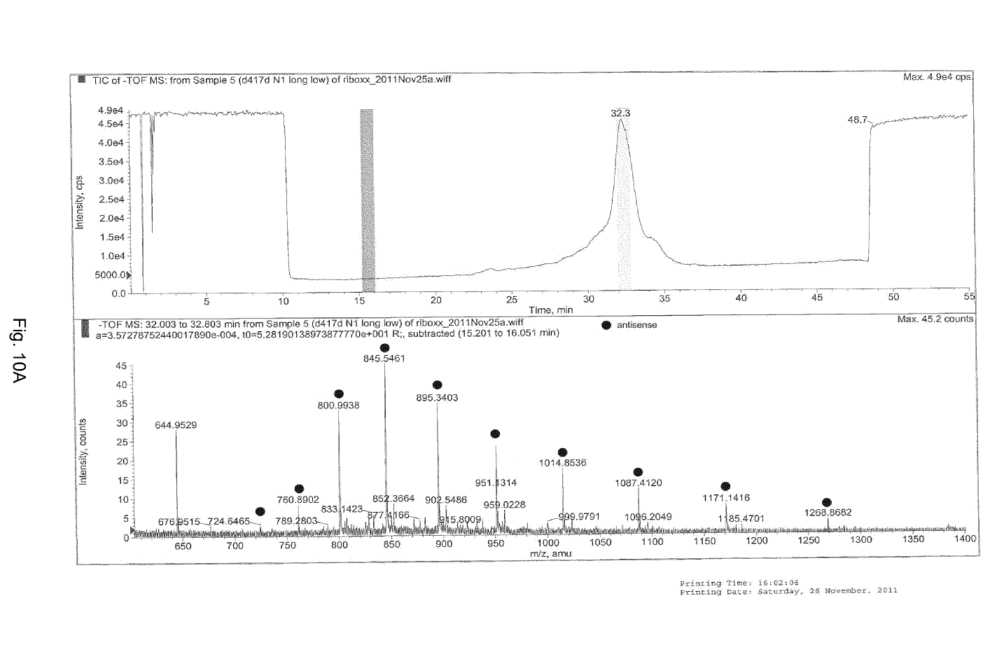

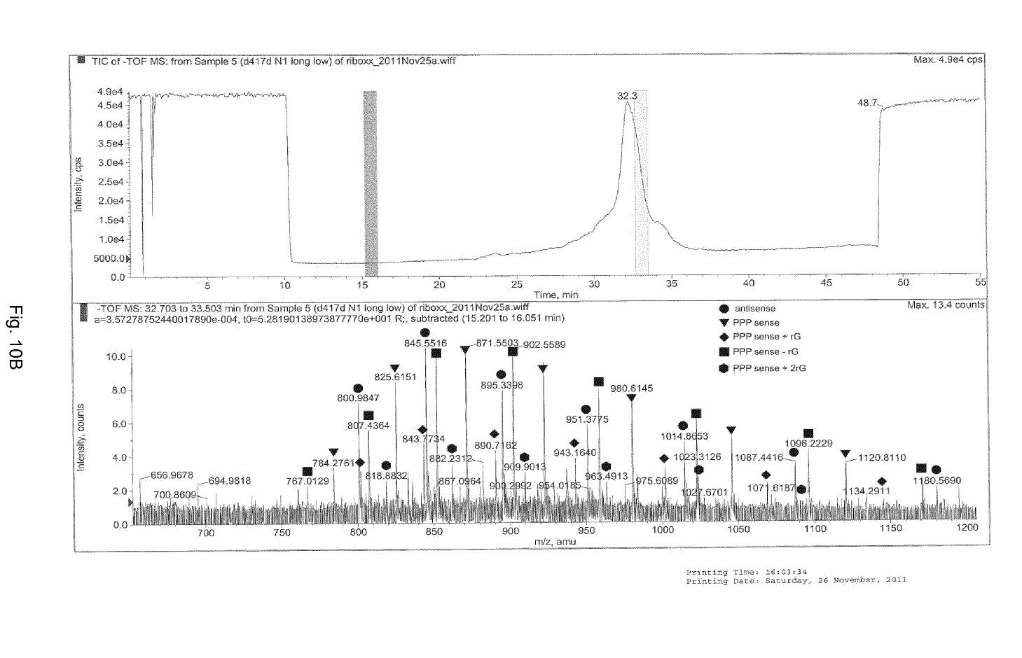

Monocytes from two different healthy donors were isolated, sorted and grown as described in example 3. Cells were then seeded in 96-well plates at a density of 25,000 cells/well in RPMI-1640 with 10% human AB serum and incubated with 6.25 μg/ml, 12.5 μg/ml, 25 μg/ml or 50 μg/ml dsRNA-90, Riboxxim®-90 or poly(I:C), respectively, for 24 to 72 hours. The concentrations of secreted of IL-1β and IL-6 in the culture supernatant were assessed using ELISA. The results are shown in FIG. 9A (IL-1β) and FIG. 9B (IL-6).

Both dsRNAs of the invention show a dose-dependent secretion of IL-1β and IL-6. The stimulation is higher for the triphosphated construct Riboxxim®-90. Poly(I:C) does not result in a dose-dependent secretion of IL-6.

The examples described above show that double-stranded RNA-molecules according to the invention trigger the secretion of cytokines such as IL-6 and IL-1β in dendritic cells and monocytes. Moreover, dsRNA of the invention containing a free 5′-triphosphate group results in a strictly dose-dependent cytokine secretion. The capability of triggering a dose-dependent immune-response makes the double-stranded RNA-molecules according to the invention highly suitable to be utilized as RNA drugs for immunostimulation or adjuvants.

1. A ribonucleic acid comprising at least one segment of double-stranded structure of at least 45 bp wherein said at least one segment of double-stranded structure has a first and a second end each having at least 3 to 10 G/C by within the last 6 to 20, respectively, by calculated from the last by of the respective end of the double-stranded structure, and wherein the nucleotide sequence between the last 6 to 20 bp at each end is heteropolymeric, wherein ribonucleic acids having the following sequences are excluded:
(SEQ ID NO: 3)
GGGUUUUUUUUUUUUUUUGGGUUUUUUUUUUUUUUUGGGUUUUUU
UUUUUUUUUGGG
(SEQ ID NO: 4)
GGGUUUGGGUUUGGGUUUGGGUUUGGGUUUGGGUUUGGGUUUGGGU
UUGGG
(SEQ ID NO: 5)
CCCUUUUUUUUUUUUUUUCCCUUUUUUUUUUUUUUUCCCUUUUUUUU
UUUUUUUCCC
(SEQ ID NO: 6)
CCCUUUCCCUUUCCCUUUCCCUUUCCCUUUCCCUUUCCCUUUCCCUU
UCCC.

2. The ribonucleic acid of claim 1 wherein the at least 3 to 10 terminal by of each end of said double-stranded structure are G/C bp.

3. The ribonucleic acid of claim 1 being essentially double-stranded.

4. The ribonucleic acid according to claim 1 comprising two polyribonucleotide strands.

5. The ribonucleic acid according to claim 1 wherein the ribonucleic acid is blunt ended on both sides.

6. The ribonucleic wherein the strands of the at least one segment of double-stranded structure have the following sequences:
5′-(G/C)ₓ(N)_{y}(G/C)_{z}-3′
3′-(G/C)ₓ(N)_{y}(G/C)_{z}-5′
wherein G/C is a G or C ribonucleotide with the proviso that when a G ribonucleotide is in a position in one strand the other strand has a C ribonucleotide in the opposite position and vice versa; N is any ribonucleotide with the proviso that the sequences of both strands are essentially complementary; each x is an integer of from 3 to 10, each y is an integer of 30 to 200, preferably 30 to 150, more preferably 30 to 100, even more preferred 30 to 50, and each z is an integer of from 3 to 10, with the proviso that x in one strand is equal to the x in the other strand, y in one strand is equal to the y in the other strand, and z in one strand is equal to the z in the other strand; and with the further proviso that the length of said double-stranded structure is from 45 to 220 bp, more preferably 45 to 150 bp, particularly preferred 45 to 90 bp, even more preferred 45 to 70 bp.

7. The ribonucleic acid of claim 6 wherein the strands of the at least one segment of double-stranded structure are one of the following sequence pairs:
5′-(G)₅(N)_{y}(G)₅-3′
3′-(C)₅(N)_{y}(C)₅-5′
or
5′-(C)₅(N)_{y}(C)₅-3′
3′-(G)₅(N)_{y}(G)₅-5′
or
5′-(G)₅(N)_{y}(C)₅-3′
3′-(C)₅(N)_{y}(G)₅-5′
or
5′-(C)₅(N)_{y}(G)₅-3′
3′-(G)₅(N)_{y}(C)₅-5′
wherein y is an integer of from 35 to 200, preferably from 35 to 150, more preferably from 35 to 100, particularly preferred from 35 to 80, even more preferred from 35 to 40.

8. The ribonucleic acid according to claim 1 wherein the G/C content of said double-stranded structure is at least 45%.

9. The ribonucleic acid of claim 8 wherein the G/C content is from 45 to 70%.

10. The ribonucleic acid of claim 9 wherein the C/C content is from 48 to 60%.

11. The ribonucleic acid according to claim 8 wherein the Tm of said double-stranded structure is from 68 to 80° C.

12. The ribonucleic acid of claim 6 wherein the strands of the at least one segment of double-stranded structure are one of the following sequence pairs:
(SEQ ID NO: 8)
5′-CCCCCUAAGCACGAAGCUCAGAGUUAAGCACGAAGCUCAGA
GUCCCCC-3′
(SEQ ID NO: 9)
5′-GGGGGACUCUGAGCUUCGUGCUUAACUCUGAGCUUCGUGCU
UAGGGGG-3′
(SEQ ID NO: 10)
5′-CCCCCGAACGAAUUUAUAAGUGGGAACGAAUUUAUAAGUG
GCCCCC-3′
(SEQ ID NO: 11)
5′-GGGGGCCACUUAUAAAUUCGUUCCCACUUAUAAAUUCGUU
CGGGGG-3′
(SEQ ID NO: 12)
5′-CCCCCACAACAUUCAUAUAGCUGACAACAUUCAUAUAGCUG
CCCCC-3′
(SEQ ID NO: 13)
5′-GGGGGCAGCUAUAUGAAUGUUGUCAGCUAUAUGAAUGUUGU
GGGGG-3′
(SEQ ID NO: 14)
5′-CCCCCUAAGCAGCAAGCCUCAGCAGCUAAGCCAGCAGCCUCAGCAG
CUAGCAGCAAGCUCAGCAGCUAAGCCACGAGCUCAUGCGCCCCC-3′
(SEQ ID NO: 15)
5′-GGGGGCGCAUGAGCUCGUGGCUUAGCUGCUGAGCUUGCUGCUAGC
UGCUGAGGCUGCUGGCUUAGCUGCUGAGGCUUGCUGCUUAGGGGG-3′

13. The ribonucleic acid according to claim 1 comprising a free triphosphate group at the 5′-end of at least one strand.

14. The ribonucleic acid of claim 13 wherein the strands are selected from the following sequence pairs:
(SEQ ID NO: 8)
5′-CCCCCUAAGCACGAAGCUCAGAGUUAAGCACGAAGCUCAGAGU
CCCCC-3′
(SEQ ID NO: 9)
ppp-5′-GGGGGACUCUGAGCUUCGUGCUUAACUCUGAGCUUCGUGC
UUAGGGGG-3′
(SEQ ID NO: 14)
5′-CCCCCUAAGCAGCAAGCCUCAGCAGCUAAGCCAGCAGCCUCAGC
AGCUAGCAGCAAGCUCAGCAGCUAAGCCACGAGCUCAUGCGCCCCC-3′
(SEQ ID NO: 15)
ppp-5′-GGGGGCGCAUGAGCUCGUGGCUUAGCUGCUGAGCUUGCUGCU
AGCUGCUGAGGCUGCUGGCUUAGCUGCUGAGGCUUGCUGCUUAGGGGG-3′
wherein ppp denotes a free triphosphate group.

15. A pharmaceutical composition comprising at least one ribonucleic acid according to claim 1 in combination with at least one pharmaceutically acceptable carrier.

16. The pharmaceutical composition of claim 15 comprising a vaccine.

17. The ribonucleic acid according to claim 1 for use as a medicament.

18. The ribonucleic acid according to claim 1 for immunostimulation.

19. Use of the ribonucleic acid according to claim 1 as an agonist of Toll-like receptor 3 (TLR-3).

20. Use of the ribonucleic acid of claim 13 as an agonist of TLR-3 and RIG-I.

21. A method of producing the ribonucleic acid according to claim 1 comprising the steps of:
(a) synthesizing a ribonucleic acid having a sequence that folds into a double-stranded structure having at least one segment of double-stranded structure of at least 45 bp wherein said at least one segment of double-stranded structure has a first and a second end each having at least 3 to 10 G/C by within the last 6 to 20, respectively, by calculated from the last by of the respective end of the double-stranded structure, and wherein the nucleotide sequence between the last 6 to 20 bp at each end is heteropolymeric, wherein ribonucleic acids having the following sequences are excluded:
(SEQ ID NO: 3)
GGGUUUUUUUUUUUUUUUGGGUUUUUUUUUUUUUUUGGGUUUUUUUUUUU
UUUUGGG,
(SEQ ID NO: 4)
GGGUUUGGGUUUGGGUUUGGGUUUGGGUUUGGGUUUGGGUUUGGGUUUG
GG
(SEQ ID NO: 5)
CCCUUUUUUUUUUUUUUUCCCUUUUUUUUUUUUUUUCCCUUUUUUUUU
UUUUUUCCC
SEQ ID NO: 6
CCCUUUCCCUUUCCCUUUCCCUUUCCCUUUCCCUUUCCCUU
UCCCUUUCCC
(b) synthesizing a first ribonucleic acid strand and a second ribonucleic acid strand and annealing the two strands to form a double stranded structure having at least one segment of double-stranded structure of at least 45 bp wherein said at least one segment of double-stranded structure has a first and a second end each having at least 3 to 10 G/C bp within the last 6 to 20, respectively, by calculated from the last by of the respective end of the double-stranded structure, and wherein the nucleotide sequence between the last 6 to 20 bp at each end is heteropolymeric, wherein ribonucleic acids having the following sequences are excluded:
(SEQ ID NO: 3)
GGGUUUUUUUUUUUUUUUGGGUUUUUUUUUUUUUUUGGGUUUUUUUUU
UUUUUUGGG
(SEQ ID NO: 4)
GGGUUUGGGUUUGGGUUUGGGUUUGGGUUUGGGUUUGGGUUUGGGUUUG
GG
(SEQ ID NO: 5)
CCCUUUUUUUUUUUUUUUCCCUUUUUUUUUUUUUUUCCCUUUUUUUUUU
UUUUUCCC
(SEQ ID NO: 6)
CCCUUUCCCUUUCCCUUUCCCUUUCCCUUUCCCUUUCCCUUUCCC
UUUCCC.


SEQUENCE LISTING (19 sequences)

SEQ ID NO: 1 - RNA, 45 nt, artificial sequences
  source 1..45: /mol_type=“RNA” /note=“Prior art RNA” /organism=“artificial sequences”
gggggggguuuuuuuuuuuuuuuuuuuuuuuuuuuuuuggggggg

SEQ ID NO: 2 - RNA, 47 nt, artificial sequences
  source 1..47: /mol_type=“RNA” /note=“Prior art RNA” /organism=“artificial sequences”
ggggggggguuuuuuuuuuuuuuuuuuuuuuuuuuuuuugggggggg

SEQ ID NO: 3 - RNA, 57 nt, artificial sequences
  source 1..57: /mol_type=“RNA” /note=“Prior art RNA” /organism=“artificial sequences”
ggguuuuuuuuuuuuuuuggguuuuuuuuuuuuuuuggguuuuuuuuuuuuuuuggg

SEQ ID NO: 4 - RNA, 51 nt, artificial sequences
  source 1..51: /mol_type=“RNA” /note=“Prior art RNA” /organism=“artificial sequences”
ggguuuggguuuggguuuggguuuggguuuggguuuggguuuggguuuggg

SEQ ID NO: 5 - RNA, 57 nt, artificial sequences
  source 1..57: /mol_type=“RNA” /note=“Prior art RNA” /organism=“artificial sequences”
cccuuuuuuuuuuuuuuucccuuuuuuuuuuuuuuucccuuuuuuuuuuuuuuuccc

SEQ ID NO: 6 - RNA, 51 nt, artificial sequences
  source 1..51: /mol_type=“RNA” /note=“Prior art RNA” /organism=“artificial sequences”
cccuuucccuuucccuuucccuuucccuuucccuuucccuuucccuuuccc

SEQ ID NO: 7 - RNA, 40 nt, artificial sequences
  source 1..40: /mol_type=“RNA” /note=“Prior art RNA” /organism=“artificial sequences”
ccccccccccccccccccccgguuuuuuuuuuuuuuuggg

SEQ ID NO: 8 - RNA, 48 nt, artificial sequences
  source 1..48: /mol_type=“RNA” /note=“Riboxxim-1 sense strand” /organism=“artificial sequences”
cccccuaagcacgaagcucagaguuaagcacgaagcucagaguccccc

SEQ ID NO: 9 - RNA, 48 nt, artificial sequences
  source 1..48: /mol_type=“RNA” /note=“Riboxxim-1/Riboxxim-48 antisense strand” /organism=“artificial sequences”
gggggacucugagcuucgugcuuaacucugagcuucgugcuuaggggg

SEQ ID NO: 10 - RNA, 46 nt, artificial sequences
  source 1..46: /mol_type=“RNA” /note=“Riboxxim-2 sense strand” /organism=“artificial sequences”
cccccgaacgaauuuauaagugggaacgaauuuauaaguggccccc

SEQ ID NO: 11 - RNA, 46 nt, artificial sequences
  source 1..46: /mol_type=“RNA” /note=“Riboxxim-2 antisense strand” /organism=“artificial sequences”
gggggccacuuauaaauucguucccacuuauaaauucguucggggg

SEQ ID NO: 12 - RNA, 46 nt, artificial sequences
  source 1..46: /mol_type=“RNA” /note=“Riboxxim-3 sense strand” /organism=“artificial sequences”
cccccacaacauucauauagcugacaacauucauauagcugccccc

SEQ ID NO: 13 - RNA, 46 nt, artificial sequences
  source 1..46: /mol_type=“RNA” /note=“Riboxxim-3 antisense strand” /organism=“artificial sequences”
gggggcagcuauaugaauguugucagcuauaugaauguuguggggg

SEQ ID NO: 14 - RNA, 90 nt, artificial sequences
  source 1..90: /mol_type=“RNA” /note=“dsRNA-90/Riboxxim-90 sense strand” /organism=“artificial sequences”
cccccuaagcagcaagccucagcagcuaagccagcagccucagcagcuagcagcaagcucagcagcuaagccacgagcucaugcgccccc

SEQ ID NO: 15 - RNA, 90 nt, artificial sequences
  source 1..90: /mol_type=“RNA” /note=“dsRNA-90/Riboxxim-90 antisense strand” /organism=“artificial sequences”
gggggcgcaugagcucguggcuuagcugcugagcuugcugcuagcugcugaggcugcuggcuuagcugcugaggcuugcugcuuaggggg

SEQ ID NO: 16 - RNA, 11 nt, artificial sequences
  source 1..11: /mol_type=“RNA” /note=“General formula of sense/antisense strand” /organism=“artificial sequences”
gggggnggggg

SEQ ID NO: 17 - RNA, 11 nt, artificial sequences
  source 1..11: /mol_type=“RNA” /note=“General formula of sense/antisense strand” /organism=“artificial sequences”
cccccnccccc

SEQ ID NO: 18 - RNA, 11 nt, artificial sequences
  source 1..11: /mol_type=“RNA” /note=“General formula of sense/antisense strand” /organism=“artificial sequences”
gggggnccccc

SEQ ID NO: 19 - RNA, 11 nt, artificial sequences
  source 1..11: /mol_type=“RNA” /note=“General formula of sense/antisense strand” /organism=“artificial sequences”
cccccnggggg